(19) United States
(12) Patent Application Publication
(10) Pub. No.: US 2025/0331706 A1
(43) Pub. Date: Oct. 30, 2025

(54) GUI ASSOCIATED WITH PRIMING A FLUID MANAGEMENT SYSTEM

(71) Applicant: BOSTON SCIENTIFIC SCIMED, INC., Maple Grove, MN (US)

(72) Inventors: Iwona Zietara, Cahir (IE); Paul Byrne, Kilkenny (IE); Elizabeth A. Stokley, Raleigh, NC (US); Teagan Jo Walter, Boston, MA (US); Anant Vaidyanathan Subramaniam, Boston, MA (US)

(73) Assignee: BOSTON SCIENTIFIC SCIMED, INC., Maple Grove, MN (US)

(21) Appl. No.: 19/193,212

(22) Filed: Apr. 29, 2025

Related U.S. Application Data
(60) Provisional application No. 63/640,509, filed on Apr. 30, 2024.

(51) Int. Cl. A61B 1/015; A61B 1/00; A61M 1/00; G16H 40/63

(52) U.S. Cl. CPC A61B 1/015; A61B 1/00006; A61B 1/0004; A61B 1/00045; A61M 1/00; G16H 40/63; A61B 2560/0462; A61B 2562/0247; A61M 2202/0007; A61M 2202/04; A61M 2205/10; A61M 2205/121; A61M 2205/3317; A61M 2205/3327; A61M 2205/3344; A61M 2205/502

(57) ABSTRACT

A fluid management system including a fluid management console and an associated graphical user interface (GUI) is disclosed. In some instances, the GUI includes a series of start-up screens including a fluid priming screen prompting a user to select between a quick set-up mode and a step-by-step setup mode in order to complete a priming procedure of the fluid cassette, the GUI includes a plurality of medical procedure mode screens including a first mode for use of the fluid management system with a pressure sensing endoscope and one or more modes for use of the fluid management system with an endoscope without a pressure sensor, and/or the GUI includes a screen to input and/or set intraluminal pressure limiter settings into the controller with a confirmation window requiring a multiple step confirmation process to confirm that the input/set intraluminal pressure limiter settings are acceptable.

### CROSS-REFERENCE TO RELATED APPLICATIONS

The application claims the benefit of U.S. Provisional Patent Application Ser. No. 63/640,509, filed on Apr. 30, 2024, the disclosure of which is incorporated herein by reference.

### TECHNICAL FIELD

The disclosure is directed to a fluid management system. More particularly, the disclosure is directed to aspects of a graphical user interface (GUI) associated with a fluid management system during start up and operation of the fluid management system.

### BACKGROUND

Flexible ureteroscopy (fURS), gynecology, and other endoscopic procedures require the circulation of fluid for several reasons. Fluid management systems may be used to deliver fluid to an anatomical cite from a reservoir at a desired pressure and/or flowrate via a peristaltic or roller pump. Fluid management systems may adjust the flowrate and/or pressure at which fluid is delivered from the reservoir based on data collected from a procedural device, such as, but not limited to, pressure readings sensed and/or obtained by the fluid management system. The fluid management system may utilize a disposable fluid tubing set installed with a pump console to provide the fluid to the patient. There is an ongoing need to provide alternative configurations of the components of fluid management systems, to facilitate the use thereof.

### BRIEF SUMMARY

This disclosure provides design, material, manufacturing method, and use alternatives for components of a fluid management system.

A first example is a fluid management system. The fluid management system includes a fluid management console. The console includes a housing, a controller housed within the housing, an inflow pump disposed within the housing, and a graphical user interface (GUI) associated with the housing. The fluid management system also includes a fluid cassette configured to be received within a receptacle of the housing of the fluid management console. The GUI includes a series of start-up screens including a fluid priming screen prompting a user to select between a quick setup mode and a step-by-step setup mode in order to complete a priming procedure of the fluid cassette.

Alternatively or additionally to any of the examples above, in another example, the quick setup mode includes a screen listing a series of steps to be performed to complete the priming procedure.

Alternatively or additionally to any of the examples above, in another example, each of the series of steps must be confirmed prior to completing the priming procedure.

Alternatively or additionally to any of the examples above, in another example, the controller confirms each of the series of steps with one or more sensors.

Alternatively or additionally to any of the examples above, in another example, the one or more sensors include one or more proximity sensors, one or more contact sensors, and/or one or more magnetic sensors.

Alternatively or additionally to any of the examples above, in another example, the quick setup mode includes a user input to confirm that a clamp on a fluid outlet tubing extending from the fluid cassette has been closed.

Alternatively or additionally to any of the examples above, in another example, confirmation that the clamp on the fluid outlet tubing is closed must be completed prior to the controller initiating the priming procedure.

Alternatively or additionally to any of the examples above, in another example, the step-by-step setup mode includes one or more screens of the GUI depicting an image of: a) opening the door the console; b) installing the fluid cassette within the receptacle of the housing; or c) closing the door.

Alternatively or additionally to any of the examples above, in another example, the image is animated.

Alternatively or additionally to any of the examples above, in another example, the step-by-step setup mode includes a user input to confirm that a clamp on a fluid outlet tubing extending from the fluid cassette has been closed.

Another example is a fluid management system. The system includes a fluid management console. The console includes a housing, a controller housed within the housing, an inflow pump disposed within the housing, and a graphical user interface (GUI) associated with the housing. The GUI includes a plurality of medical procedure mode screens for setting one or more parameters for controlling the inflow pump. The plurality of medical procedure mode screens includes a first mode for use of the fluid management system with a pressure sensing endoscope having a pressure sensor incorporated therewith for communicating intraluminal pressure to the controller, and one or more modes for use of the fluid management system with an endoscope without a pressure sensor.

Alternatively or additionally to any of the examples above, in another example, the plurality of medical procedure mode screens includes a cystoscopy mode and a ureteroscopy mode.

Alternatively or additionally to any of the examples above, in another example, upon selecting the ureteroscopy mode, the GUI includes a prompt and/or window permitting the user to start the medical procedure in the cystoscopy mode.

Alternatively or additionally to any of the examples above, in another example, the controller includes different user selectable pump pressure settings in the cystoscopy mode than in the ureteroscopy mode.

Alternatively or additionally to any of the examples above, in another example, upon selecting the first mode, the GUI includes a prompt and/or window permitting the user to start the medical procedure in a cystoscopy mode.

Alternatively or additionally to any of the examples above, in another example, the controller includes different user selectable pump pressure settings in the cystoscopy mode than in the first mode.

Another example is a fluid management system. The fluid management system includes a fluid management console. The console includes a housing, a controller housed within the housing, an inflow pump disposed within the housing, and a graphical user interface (GUI) associated with the housing. The GUI includes a medical procedure mode screen for setting one or more parameters for controlling the inflow pump for use of the fluid management system with a pressure sensing endoscope having a pressure sensor incorporated therewith. The GUI includes a screen to input and/or set intraluminal pressure limiter settings into the controller.

Alternatively or additionally to any of the examples above, in another example, the GUI includes a confirmation window requiring a multiple step confirmation process to confirm that the input/set intraluminal pressure limiter settings are acceptable.

Alternatively or additionally to any of the examples above, in another example, the GUI includes an intraoperative screen permitting a user to adjust the intraluminal pressure limiter settings during a medical procedure.

Alternatively or additionally to any of the examples above, in another example, the intraluminal pressure limiter settings include an intraluminal pressure limiter setting for fluid flow through an attached endoscope and an intraluminal pressure limiter setting for fluid flush through the attached endoscope a fluid flush is activated by the attached endoscope, the intraluminal pressure limiter setting for fluid flow being independent of the intraluminal pressure limiter setting for fluid flush.

Another example is a fluid management system. The fluid management system includes a fluid management console. The console includes a housing, a controller housed within the housing, an inflow pump disposed within the housing, and a graphical user interface (GUI) associated with the housing. The GUI includes a plurality of medical procedure mode screens for setting one or more parameters for controlling the inflow pump. The plurality of medical procedure mode screens includes a cystoscopy mode and a ureteroscopy mode. Upon selecting the ureteroscopy mode, the GUI includes a prompt and/or window permitting the user to start the medical procedure in the cystoscopy mode.

Alternatively or additionally to any of the examples above, in another example, the controller includes different user selectable pump pressure settings in the cystoscopy mode than in the ureteroscopy mode.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set; and

FIGS. 3-22 depict aspects of a graphical user interface (GUI) associated with a fluid management system during start up and operation of the fluid management system.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may control the flow of fluid into the body cavity and/or regulate body cavity pressure and/or the flowrate of fluid flow to the body cavity using an inflow and/or outflow pump of the fluid management system. The inflow pump may deliver fluid through inflow tubing of a fluid tubing set to the patient and/or the outflow pump may remove fluid through outflow tubing of a fluid tubing set from the patient. The fluid management system may include one or more sensors providing signals to the controller of the fluid management system to control the fluid flow.

In some instances, in which the fluid management system is used in conjunction with an endoscope device such as, but not limited to, a LithoVue™ Elite endoscope, the fluid management system may control the fluid flow using pressure and/or temperature data from the endoscope or other endoscopic device. Direct regulation of the intraluminal pressure during a medical procedure using a pressure sensor on the endoscope may allow the fluid management system to safely control the fluid pressure with the body cavity.

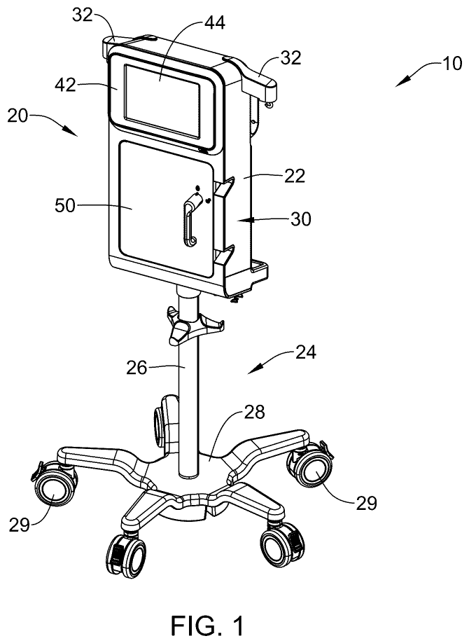

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the Litho Vue™ Elite endoscope, or other endoscope. In some instances, the endoscope may include a temperature sensor to provide intraluminal temperature feedback to the fluid management system 10, a pressure sensor to provide intraluminal pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms (such as, but not limited to, a max pressure alarm), information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate or the fluid pressure based on previously set parameters, as discussed herein.

An illustrative fluid management unit may include one or more fluid container supports, such as fluid supply source hanger(s) 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

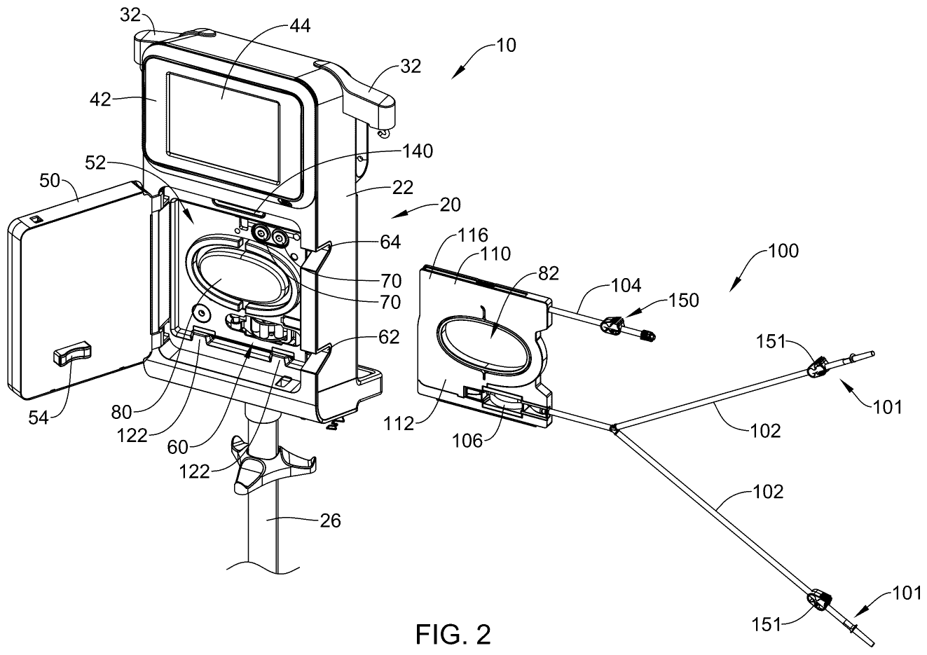

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. As noted herein, the inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target fluid flowrate or target fluid pressure based on a set of system operating parameters. In some embodiments, the controller 30 may be configured to control the inflow pump 60 to maintain a desired fluid pressure at the treatment site or a desired flowrate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may the adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike 101, or other connector for connecting to the fluid supply source(s). For example, each of the first and second tubing sections of the inflow tubing 102 may include a bag spike 101 configured to be inserted into (e.g., spiked) int a fluid bag (e.g., saline) for use during a medical procedure. Clamps may be positioned along the inflow tubing 102, which may be selectively opened and closed to control fluid flow from the fluid bags through the inflow tubing 102. The single use tubing set 100 may also include outflow tubing 104 providing a fluid outflow from the interior of the cassette 110 to a medical device connected thereto. A clamp 150 may be positioned along the outflow tubing 104, which may be selectively opened and closed to control flow from the fluid cassette 110 through the outflow tubing 104. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

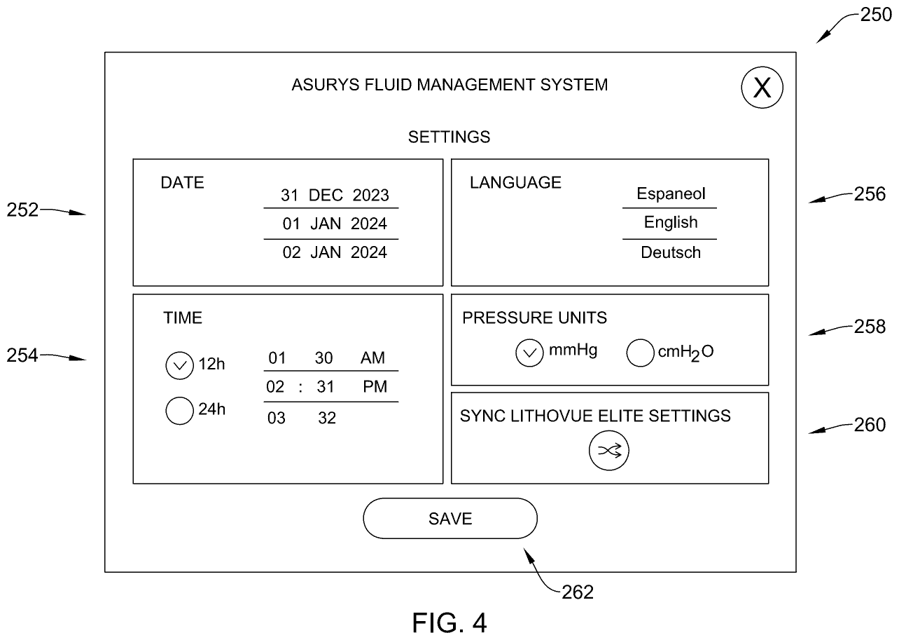

The console 20 may further include one or more additional sensors, such as a pressure sensor and/or a bubble sensor. For instance, the console 20 may include a pressure sensor 70, illustrated as a pair of pressure sensors, configured to monitor a system pressure of fluid exiting the cassette 110 and flowing through the outflow tubing 104 to a surgical site. The fluid cassette 110 may include a corresponding pressure sensor interface 72, such as a flexible membrane, (shown in FIG. 4) that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44.

The fluid cassette 110 of the fluid tubing set 100 may include a housing 112 defining a fluid pathway through an interior of the housing 112. When loaded in the receptacle 52 the fluid cassette 110 may include a front face configured to face the door 50 of the console 20 and a rear face configured to face a rear wall of the receptacle 52 of the console 20. Both, the inflow tubing 102 and the outflow tubing 104 may extend from a first side edge of the fluid cassette 110 to pass out through the channel 62 and the channel 64, respectively. The housing 112 of the fluid cassette 110 may also include an opening 82, such as an oval opening, extending through the housing 112 from the front face to the rear face of the fluid cassette 110. The opening 82 may extend a majority of the length of the housing 112 (i.e., a majority of the distance between the lateral edges of the housing 112) and/or a majority of the height of the housing 112 (i.e., a majority of the distance between the upper edge and the lower edge of the housing 112), in some instances. The opening 82 may be configured to receive an elevated portion of the rear wall of the receptacle 52, shown in FIG. 2 as the fluid warming system 80. The elevated portion of the rear wall of the receptacle 52 may be an oval shape sized to fit through the oval shaped opening 82 of the housing 112 of the fluid cassette 110 when the fluid cassette 110 is in its loaded position in the receptacle 52. In embodiments, in which the console 20 lacks a fluid warming system, the elevated portion of the rear wall of the receptacle 52 may still be present. Insertion of the elevated portion of the rear wall of the receptacle 52 through the opening 82 of the fluid cassette 110 may facilitate proper alignment of the fluid cassette 110 in the receptacle 52, for example.

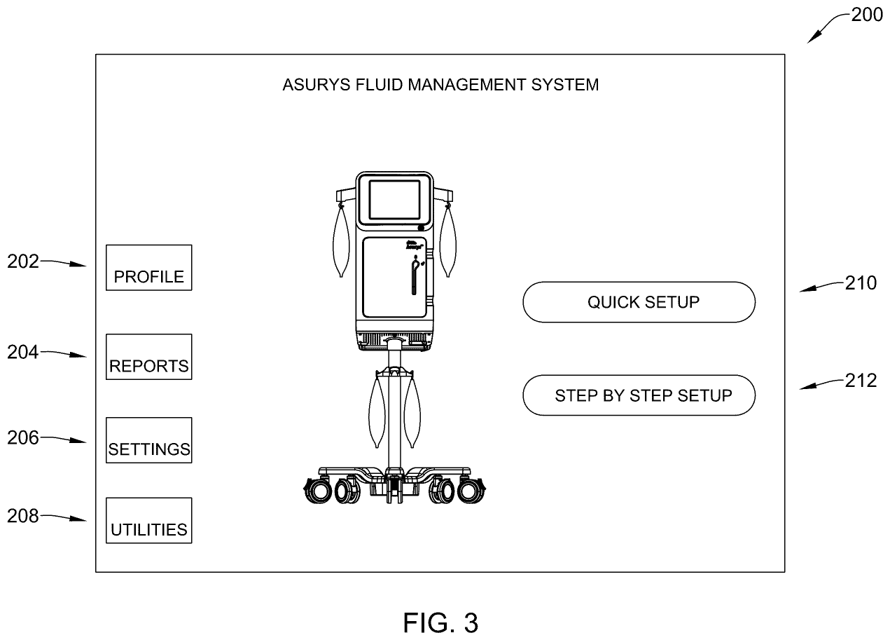

FIGS. 3-22 depict aspects of a graphical user interface (GUI) associated with a fluid management system during start up and operation of the fluid management system. As shown in FIG. 3, once the fluid management system has been powered on, a start-up screen, or Home Screen 200, may be displayed on the display screen 44 of the user input interface 42. The Home Screen 200 may include one or more user-selectable features (e.g., icons) to permitting a user to input data, select options, set actions, review reports, etc. For example, the Home Screen 200 may include a user-selectable feature (e.g., icon) 202 to set/select a user profile associated with a medical procedure to be performed with the fluid management system 10. For example, the user-selectable feature 202 may permit the user to select from one or more stored user profiles and/or may permit the user to set a new user profile. The user profile may permit the user to set various parameters (e.g., flow limits, pressure limits, target flow rate, target intraluminal pressure, etc.) default parameters for the specific user profile selected.

The Home Screen 200 may also include a user-selectable feature (e.g., icon) 204 to select/view a report associated with a previous medical procedure performed with the fluid management system 10. The report may include recorded data, such as the user profile associated with the medical procedure, date and/or time of the medical procedure, duration of the medical procedure, pressures experienced during the medical procedure, flow rates experienced during the medical procedure, any alarm notifications occurring during the medical procedure, etc.

The Home Screen 200 may also include a user-selectable feature (e.g., icon) 206 to select/input settings for use during a medical procedure and/or a user-selectable feature (e.g., icon) 208 to access other utilities available. For example, the Utilities icon 208 may be used to display the model number and/or serial number of the console 20, current software version of the fluid management system and/or prompts/protocol for updating the software, etc.

When the Settings icon 206 is selected, a Settings Screen 250, shown in FIG. 4, may be displayed on the display screen 44 of the user input interface 42. The Settings Screen 250 may include a GUI input 252 to prompt/permit a user to set/confirm the date and a GUI input 254 to prompt/permit a user to set/confirm the time of day, such that an accurate date/time of a medical procedure may be recording for a medical procedure performed with the fluid management system 10. The Settings Screen 250 may also include a GUI input 256 to set/confirm the language that information is displayed/recorded. The GUI input 256 may include a drop-down menu, a scrolling menu, etc. including a plurality of languages to select from. The Settings Screen 250 may also include a GUI input 258 to select the units of measurement in which the pressure readings are displayed/recorded. For instance, the user may select between mmHg and cmH₂O for the units of pressure to be displayed/recorded. The Settings Screen 250 may also include a GUI input 260 that the user may select to sync the settings of the fluid management system 10 with the settings of an attached endoscope (e.g., LithoVue™ Elite endoscope). For example, the time & date, language, and units of pressure may be synced with a user console of a connected endoscope. Once the desired settings have been input/confirmed, the user may select the “Save” button 262 to store the settings and return to the start-up screen 200.

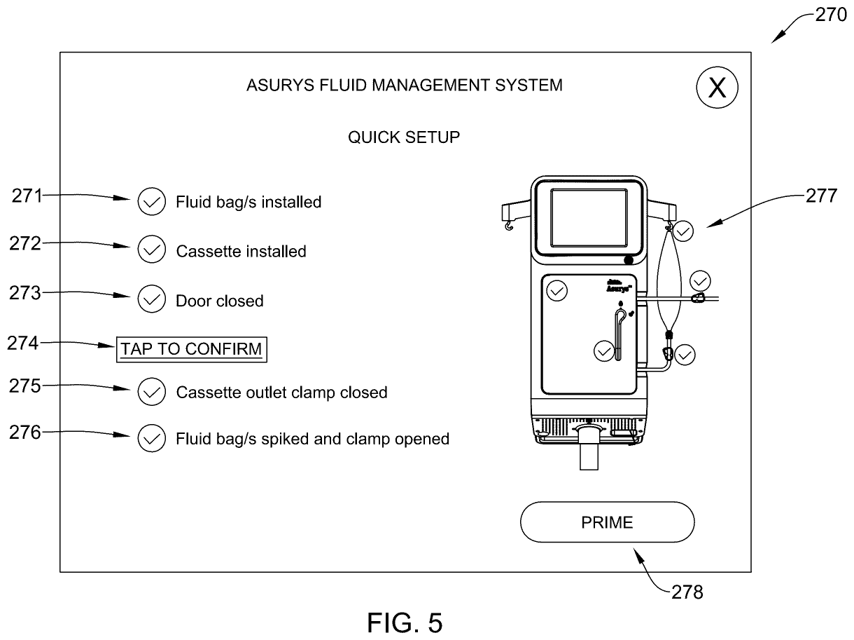

Returning to the Home Screen 200 of FIG. 2, in order to begin or initiate a medical procedure with the fluid management system 10, the user can select between a first user-selectable feature (e.g., icon, button) to begin a “Quick Setup” 210 (e.g., a quick setup mode) and a second user-selectable feature (e.g., icon, button) to begin a “Step-by-Step Setup” 212 (e.g., a step-by-step setup mode). If the Quick Setup button 210 is selected, the GUI will display the Quick Setup screen 270, shown at FIG. 5, on the display screen 44 of the user input interface 42 and initiate the quick setup mode. The Quick Setup screen 270 includes a list of steps that must be performed prior to beginning a medical procedure. The steps include a “Fluid bag/s installed” notification 271, a “Cassette installed” notification 272, and a “Doors closed” notification” 273. Once the fluid management system 10 detects that a fluid bag has been hung on the fluid supply source hanger(s) 32 via the load cell, a notification signal may be generated (e.g., the check box may be illuminated) next to the “Fluid bag/s installed” notification 271. Additionally, once the fluid management system 10 detects that a fluid cassette 110 has been installed in the receptacle 52 of the console 20, a notification signal may be generated (e.g., the check box may be illuminated) next to the “Cassette installed” notification 272. For example, the console 20 may include one or more proximity sensors, contact sensors, magnetic sensors, or the like to determine whether the fluid cassette 110 is properly positioned in the receptacle 52 prior to moving past the Quick Setup screen 270 and permitting the controller 30 to start the inflow pump 60. Furthermore, once the fluid management system 10 detects that the door 50 has been properly closed, a notification signal may be generated (e.g., the check box may be illuminated) next to the “Door closed” notification 273. For example, the console 20 may include one or more proximity sensors, contact sensors, magnetic sensors, or the like to determine whether the door 50 has been properly closed prior to moving past the Quick Setup screen 270 and permitting the controller 30 to start the inflow pump 60.

Once each of these steps have been completed/confirmed (e.g., with the associated illuminated check boxes), the user may the “Tap to Confirm” button 274 to confirm and move on to the next step in the system set-up process, which is priming the fluid management system.

Prior to any interaction between the fluid management system 10, the attached endoscope, and the patient, the fluid cassette 110 and outflow tubing 104 may be primed. Priming refers to filling the fluid cassette 110 and outflow tubing 104 with fluid to remove air before a procedure begins. Priming may be necessary because air bubbles that may be introduced during a procedure can impair visibility. In some cases, it may be desirable to complete the priming procedure in more than one stage when setting up the fluid management system 10 prior to a procedure. For example, it is contemplated that one person can prime the fluid cassette 110 while another person sets up the outflow tubing 104 with the endoscope. However, the fluid management system 10 may need to be able to maintain the fluid cassette 110 in a primed configuration while the outflow tubing 104 is being set up and the physician is preparing for the procedure. Then, once the endoscope is connected and everything is ready, a second priming stage can be started to push the air out of the outflow tubing 104 and the endoscope if so desired. In some cases, the second stage of the priming procedure may be omitted. However, performing the second stage of the priming procedure may reduce the amount of time required to begin a procedure when the physician is ready. It is contemplated that performing the priming procedure incrementally or in more than one stage may allow for flexibility when setting up the fluid management system 10 before a procedure and/or may reduce down time for the physician prior to starting a procedure.

The fluid management system 10 may be configured to include a two-stage priming procedure. In some cases, the first stage of the priming procedure may occur or be performed up to one hour before the second stage of the priming procedure. This is just one example. In other cases, the time delay between the completion of the first stage and the initialization of the second stage may be less than one hour or greater than one hour. Generally, the first stage of the priming procedure may purge air from the fluid cassette 110. During the first stage of the priming procedure, the inflow pump 60 pulls fluid from the fluid source (e.g., fluid bag) into the fluid cassette 110 via the inflow tubing 102 and the pump tubing 106, pushing the air out of the fluid path through an air vent valve or other air vents located at or near the top of the fluid cassette 110.

Prior to priming the fluid cassette 110, the user may verify that the clamp 150 (see, for example, FIG. 2) secured to the outflow tubing 104 is closed to prevent a flow of fluid through the outflow tubing 104 and a notification signal may be generated (e.g., the check box may be illuminated) next to the “Cassette outlet clamp closed” notification 275. The clamp 150 may be movable between a closed configuration configured to block a flow of fluid through the outflow tubing 104 and an open configuration configured to allow a flow of fluid through the outflow tubing 104. The closed clamp 150 may allow fluid to accumulate within the fluid cassette 110 and allow the pressure to increase within the fluid cassette 110. Furthermore, the user may verify that the fluid bag/s have been spiked with the spikes 101 of the inflow tubing 102 and the clamps 151 are opened to allow fluid flow through the inflow tubing 102, and a notification signal may be generated (e.g., the check box may be illuminated) next to the “Fluid bag/s spiked and clamp opened” notification 276. Once these steps have been confirmed, the user may select the “PRIME” button 278 to initiate the first stage of the priming procedure.

The Quick Setup screen 270 may include an image 277 illustrating aspects of the steps associated with the priming procedure. For example, the image 277 may depict the console 20 of the fluid management system with one or more icons (e.g., arrows, numbers, letters, check marks, etc.) proximate a location on the console, fluid bag, fluid tubing or otherwise associated with each of the steps to be performed during the priming procedure. For instance, the icons may be check marks that may be illuminated and/or change color once the step associated with the relevant process step has been completed. In some instances, the image 277 may include an icon (e.g., a checkmark) associated with each of the “Fluid bag/s installed” notification 271, “Cassette installed” notification 272, “Doors closed” notification” 273, “Cassette outlet clamp closed” notification 275, and “Fluid bag/s spiked and clamp opened” notification 276. In some instances, the image 277 may include animated icons depicting items to check or complete during the steps associated with the priming process.

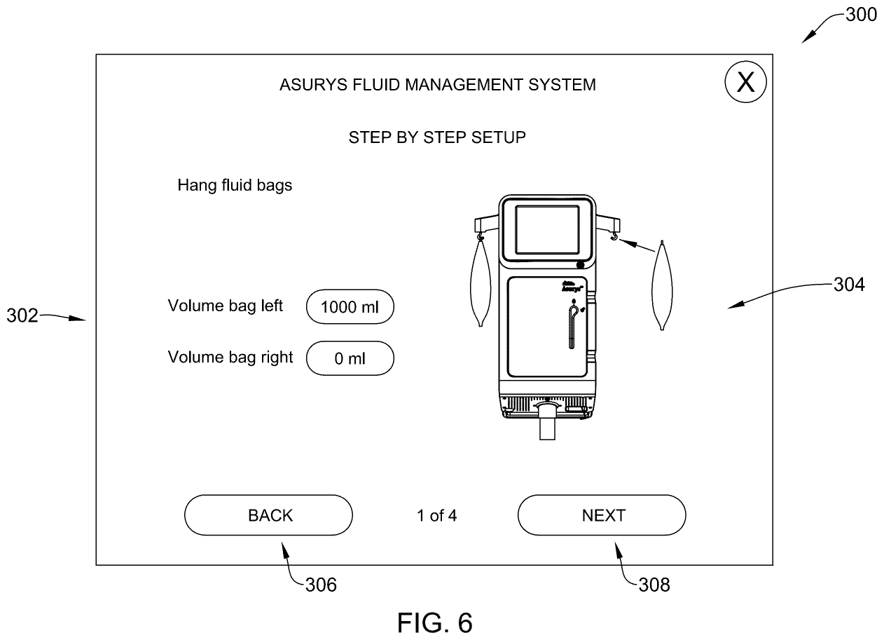

Returning to the Home Screen 200, alternatively, the user may select the “Step By Step Setup” button 212, to initiate a step-by-step tutorial walking the user through the necessary steps to prepare the fluid management system 10 for a medical procedure. Initiating the “Step By Step Setup” mode may sequentially display a series of guided GUI screens associated with each of the notifications 271-276, discussed above, to guide the user through the set-up process. For instance, a first guided set-up screen 300 (e.g., Hang fluid bags screen), shown at FIG. 6, may guide the user to hang one or more fluid bags on the fluid supply source hanger(s) 32. The fluid management system 10 may detect that a fluid bag has been hung on the fluid supply source hanger(s) 32 and display a current volume of fluid in the fluid bag on the Hang fluid bags screen 300 at 302. The fluid management system 10 may determine the presence of a fluid bag and the volume of fluid in the fluid bag via the load cell associated with the associated fluid supply source hanger(s) 32. The Hang fluid bags screen 300 may also include an image 304 of the console, depicting placement of the fluid bags on the fluid supply source hangers. In some instances, the image 304 may be animated, such that the image 304 shows movement of one or more fluid bags moving toward the console and being hung from a fluid supply source hanger. Once a fluid bag with sufficient volume of fluid has been detected, the user may select the NEXT button 308 to move to the next screen of the step-by-step setup of the GUI. In the event the user wants to return to a previous screen of the step-by-step setup of the GUI, the user may select the BACK button 306.

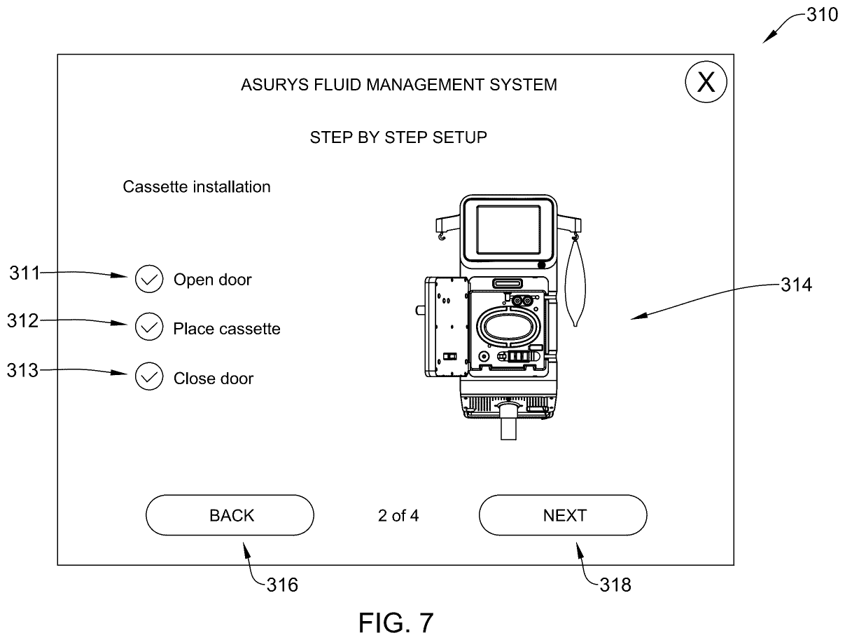

A second guided set-up screen 310 (e.g., Cassette installation screen), shown at FIG. 7, may guide the user to install a fluid cassette 110 in the receptacle 52 of the console 20 and subsequently close the door 50. The fluid management system 10 may detect and provide a notification of completion of a series of steps to be performed, including a step of opening the door 50, depicted at 311, a step of installing the fluid cassette 110 into the receptacle 52, depicted at 312, and a step of closing the door 50, depicted at 313. A notification signal may be generated (e.g., the check box may be illuminated) next to each step 311-312 upon completion and confirmation by the fluid management system 10. For example, the console 20 may include one or more proximity sensors, contact sensors, magnetic sensors, or the like to determine whether the door 52 is open, whether the fluid cassette 110 is properly positioned in the receptacle 52, and whether the door 50 has been subsequently properly closed. The Cassette installation screen 310 may also include an image 314 of the console, depicting opening the door, installing the fluid cassette, and subsequently closing the door. In some instances, the image 314 may be animated, such that the image 314 shows movement of the door and a fluid cassette moving toward the console and being installed in the console. Once a fluid cassette 110 has been properly installed and the door has been properly closed, the user may select the NEXT button 318 to move to the next screen of the step-by-step setup of the GUI. In the event the user wants to return to a previous screen of the step-by-step setup of the GUI, the user may select the BACK button 316.

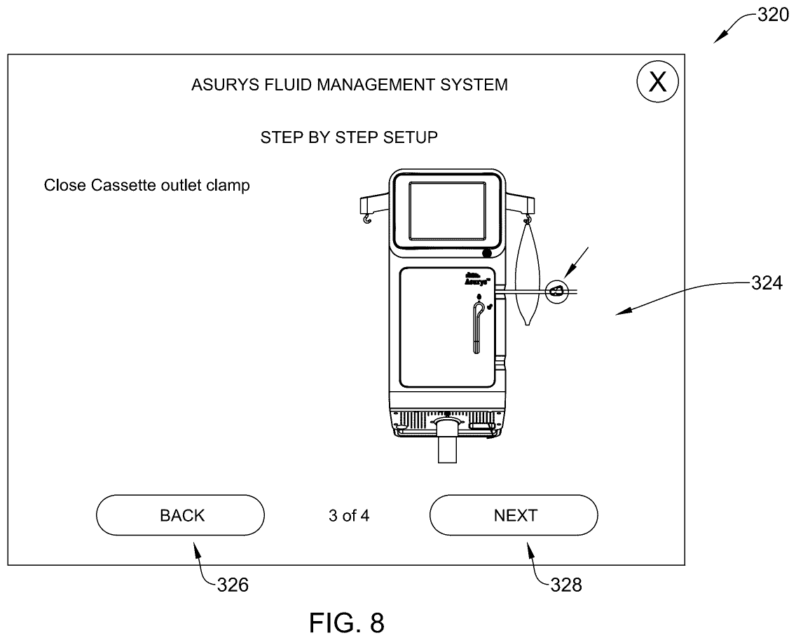

A third guided set-up screen 320 (e.g., Close cassette outlet clamp screen), shown at FIG. 8, may guide the user to close the clamp 150 on the outflow tubing 104. The closed clamp 150 may allow fluid to accumulate within the fluid cassette 110 and allow the pressure to increase within the fluid cassette 110 once the inflow pump 60 is activated. The Close cassette outlet clamp screen 320 may also include an image 324 of the console, depicting closing the clamp 150. For example, the image 324 may include an arrow pointing to the clamp in the image. In some instances, the image 324 may be animated, such that the image 324 shows the arrow flashing at the clamp and/or movement of the clamp to a closed position. Once the user has closed the clamp 150, the user may select the NEXT button 328 to move to the next screen of the step-by-step setup of the GUI. In the event the user wants to return to a previous screen of the step-by-step setup of the GUI, the user may select the BACK button 326.

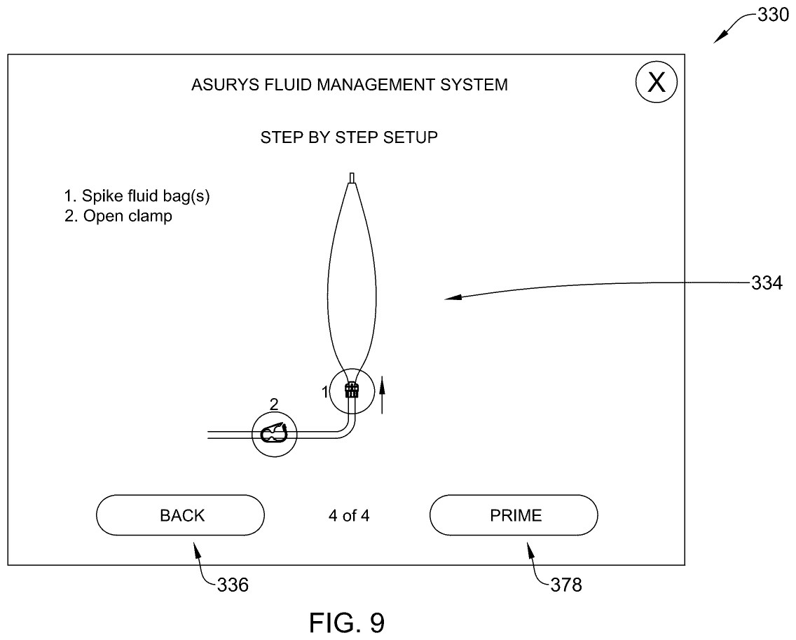

A fourth guided set-up screen 330 (e.g., Spike fluid bag/s-Open clamp screen), shown at FIG. 9, may guide the user to spike the fluid bag/s with the spikes 101 of the inflow tubing 102 and open the clamps 151 on the inflow tubing 102 to allow fluid flow through the inflow tubing 102. As shown in the Spike fluid bag/s-Open clamp screen 330, the screen may guide a user to sequentially spike the fluid bag(s) as a first step, labeled (1), followed by opening the clamp(s), labeled (2). The Spike fluid bag/s-Open clamp screen 330 may also include an image 334 of a fluid bag, depicting a spike 101 being inserted into the fluid bag. The image may be labeled with a (1) near the spike, representing this step should be completed first, and the image may be labeled with a (2) near the clamp, representing this step should be completed second. In some instances, the image 334 may include an arrow pointing to the clamp in the image. In some instances, the image 334 may be animated, such that the image 334 shows movement of the spike into the fluid bag and/or the arrow flashing at the clamp. Once the user has spiked the appropriate bag(s) and opened the clamp(s), the user may select the PRIME button 378 to begin the priming procedure. In the event the user wants to return to a previous screen of the step-by-step setup of the GUI, the user may select the BACK button 336.

Upon selecting the “PRIME” button 278 on the Quick Setup screen 270 of the Quick Setup mode or the “PRIME” button 378 on the guided set-up screen 330 (e.g., Spike fluid bag/s-Open clamp screen) of the Step By Step Setup mode, the fluid management system may initiate the first stage of the priming procedure.

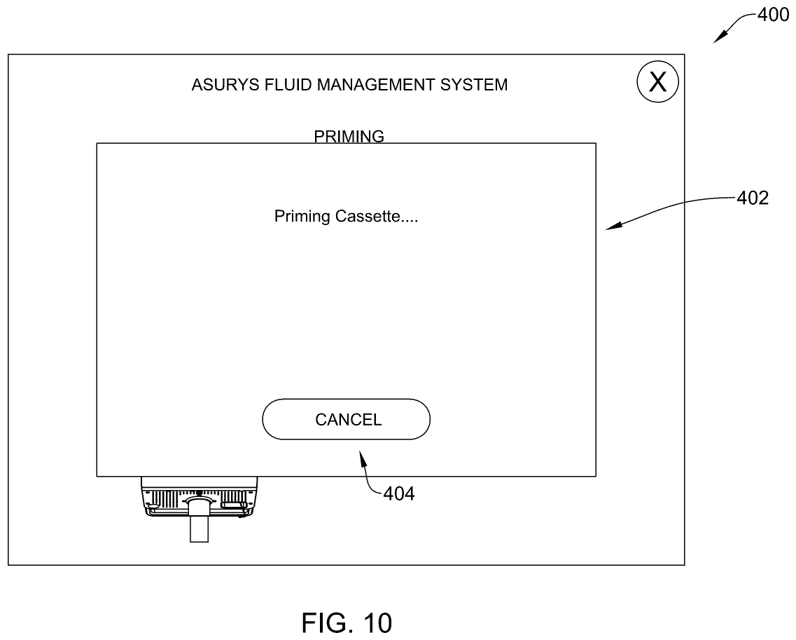

Once the “PRIME” button 278 or the “PRIME” button 378 is selected, the controller may initiate the first stage of the priming procedure and start the inflow pump 60 to pump fluid from the fluid source (e.g. fluid bag(s)) into the fluid cassette 110. As shown at FIG. 10, the GUI may include a Priming Cassette screen 400 with a pop-up window 402 notifying the user that the first stage of the priming procedure is being performed. The pop-up window 402 may include a CANCEL button 404 which the user may use to cancel the priming procedure, if needed. This first stage of the priming procedure may purge air from the fluid cassette 110. During the first stage of the priming procedure, the inflow pump 60 pulls fluid from the fluid source (e.g., fluid bag) into the fluid cassette 110 via the inflow tubing 102 and the pump tubing 106, pushing the air out of the fluid path through an air vent valve or other air vents located at or near the top of the fluid cassette 110. Because the clamp 150 is closed, fluid pressure builds with the fluid cassette 110, filling the fluid cassette 110 with fluid from the fluid source(s) while pushing air out of the fluid cassette 110, but fluid does not flow to the endoscope connected to the outflow tubing 104 of the fluid management system 10.

Once the air is removed from the fluid path, the pressure within the flow path of the fluid cassette 110 quickly increases. The first stage of the priming procedure may be successfully completed and stops the inflow pump 60 when the system pressure reaches a threshold system pressure, such as 360 mmHg.

In some embodiments, the fluid management system 10 may be configured to perform pressure sensor checks and zeroing prior to performing the first stage of the priming procedure. As noted above, the fluid management system 10 may contain two system pressure sensors 70 to detect fluid system pressure within the cassette 110. The two system pressure sensors 70 may be used to check one another. For example, once the fluid cassette 110 is inserted into the console 20 and the door 50 is closed, the cross-checking may begin. During the pressure sensor checks, a pressure sensor that is out of range (e.g., ±30 mmHg for 500 milliseconds) may considered a critical failure. An alert or error message may be displayed to the user via the touch screen interface 42. The error message may be an alphanumeric message, an audio alert, another type of visual alert, or the like. In some embodiments, the pressure sensor startup cross check may use the priming pressure at the start of priming and then the high pressure as the only pressure sensor cross check. Said differently, the pressure sensor cross check may occur during the priming procedure and utilize pressure measurements from the priming procedure as the cross check.

Zeroing the pressure sensors may also be performed before or at the start of the priming procedure. If the zeroing one or more of the pressure sensors fails (e.g., the offset needed to set the pressure sensor(s) to zero is greater than a predetermined threshold), then the priming procedure may not be performed. In some cases, the door closure dynamics may have an impact on the time to zero, zeroing may be performed prior the start of the priming. This may allow sufficient time for the debounce generated by the door 50 closing to settle down before zeroing. Zeroing may be required before the fluid managements system 10 uses the pressure sensors 70 to control the priming procedure and/or to control a medical procedure. In some embodiments, it may be desirable to zero the pressure sensors 70 after the first and/or second stage of the priming procedure has been completed. However, performing the zeroing procedure with fluid in the fluid flow path of the fluid cassette 110 may require characterization of the system and/or tight tolerances in the console 20, fluid cassette 110, and/or the priming procedure.

When the first stage of the priming is initiated, the controller 30 may start a timer. The controller 30 may continually compare a cycle time or a length of time that has elapsed since initiation of the first stage of the priming procedure with a predetermined maximum cycle length to determine if a timeout has occurred. For example, the first stage of the priming procedure must be completed within a predetermined length of time. If the first stage of the priming procedure takes too much time or exceeds the predetermined length of time, a timeout error may be issued and the first stage of the priming procedure stopped. The timeout error may occur at any point during the first stage of the priming procedure if the time elapsed exceeds the predetermined maximum allowable completion time. The timeout error may be an alphanumeric message displayed on the touch screen interface 42, an audio alert, other visual alert, or the like. If the elapsed time has not exceeded a predetermined maximum length of time, the first stage of the priming procedure may continue.

Further, the controller 30 may be configured to continually monitor the system pressure and/or the pressure within the flow path of the fluid cassette 110 with the pressure sensors 70. The controller 30 may compare a current pressure to a previous pressure to determine if the pressure has increased and/or dropped. For example, the controller 30 may be configured to obtain pressure measurements at predetermined time intervals. The controller 30 may compare a most recently acquired pressure measurement with the pressure measurement immediately preceding the current pressure measurement. If the current pressure measurement is less than the preceding pressure measurement, a pressure drop error may be issued and the first stage of the priming procedure stopped. A pressure drop that occurs within the flow path of the fluid cassette 110 may be indicative of a fluid leak in the flow path. Pressure drop errors may be generated at any time during the priming procedure if the pressure decreases by a predetermined amount or at any pressure decrease. The pressure drop error may be an alphanumeric message displayed on the touch screen interface 42, an audio alert, other visual alert, or the like. If the pressure does not lower or drop, the first stage of the priming procedure may continue.

When the first stage of the priming procedure is initiated, the controller 30 may be configured to activate the inflow pump 60 and increase the revolutions per minute (RPM) thereof until a maximum RPM is obtained to start a flow of fluid through the system and raise the pressure within the flow path of the fluid cassette 110. It is contemplated that the controller 30 may control the RPM (e.g., increase and/or decrease the RPM) of the inflow pump 60 to obtain a predetermined pressure. The controller 30 may continually monitor and store a current peak pressure measurement. In some cases, the pressure measurement may be updated at predetermined time intervals. The controller 30 may be configured to compare the current pressure measurement with a predetermined maximum pressure to determine if the maximum pressure has been met or exceeded. In some embodiments, the maximum pressure may be about 600 mmHg. This is just an example. The maximum pressure may be less than or greater than 600 mmHg, as desired. If the system pressure meets or exceeds the predetermined maximum pressure, the controller 30 may issue a high-pressure error. High-pressure errors may occur at any time during the first stage of the priming procedure when the measured pressure mects or exceeds the predetermined maximum pressure. The high-pressure error may be an alphanumeric message displayed on the touch screen interface 42, an audio alert, other visual alert, or the like. A high-pressure may be indicative of a block or obstruction in the flow path of the fluid cassette 110. If the pressure does not exceed a predetermined maximum, the first stage of the priming procedure may continue.

As the controller 30 is increasing or ramping up the RPM of the inflow pump 60, the controller 30 may be configured to compare the current peak pressure to a predetermined stop pressure to determine if the stop pressure has been reached. In some cases, the stop pressure may be about 360 mmHg. This is just an example. The stop pressure may be less than or greater than 360 mmHg, as desired. If the stop pressure has not been reached, the controller 30 may continue to increase the RPM of the inflow pump 60 until the maximum RPM is reached. If the stop pressure is not reached, an alert may be displayed on the touch screen interface 42. The “pressure not achieved” error may be an alphanumeric message displayed on the touch screen interface 42, an audio alert, other visual alert, or the like.

If the stop pressure has been reached, the controller 30 may compare a current runtime (e.g., a length of time the first stage of the priming procedure has been in process) to a predetermined minimum runtime to determine if the minimum runtime has been reached. If the minimum runtime has not been reached, the controller 30 may set the inflow pump 60 to a fixed RPM, and allow the inflow pump 60 to run until the minimum runtime has been reached. When the minimum runtime has been reached, a successful first stage of the priming procedure has been completed and the inflow pump 60 may be stopped. The controller 30 may initiate a timer and wait for the second stage of the priming procedure to begin.

It is contemplated that the timer may be countdown timer which begins at a maximum allowable time between the first stage of the priming procedure and the second stage of the priming procedure. For example, in some cases, the fluid management system 10 can wait up to 60 minutes between the completion of the first stage of the priming procedure and the start of the second stage of the priming procedure. Prior to initiating the second stage of the priming procedure, the controller 30 may be configured to determine if the timer has expired or the maximum allowable time between stages has been surpassed. In one example, if sixty minutes or more elapses between the completion of the first stage of the priming procedure and the start of the second stage of the priming procedure, the controller 30 may be configured to display a timeout error and the first stage of the priming procedure may have to be recompleted or performed again. In some embodiments, the maximum allowable time between the first stage of the priming procedure and the second stage of the priming procedure may be less than sixty minutes or more than sixty minutes, as desired. It is further contemplated that the timer may count up and compare the elapsed time to the maximum allowable time between the first stage of the priming procedure and the second stage of the priming procedure.

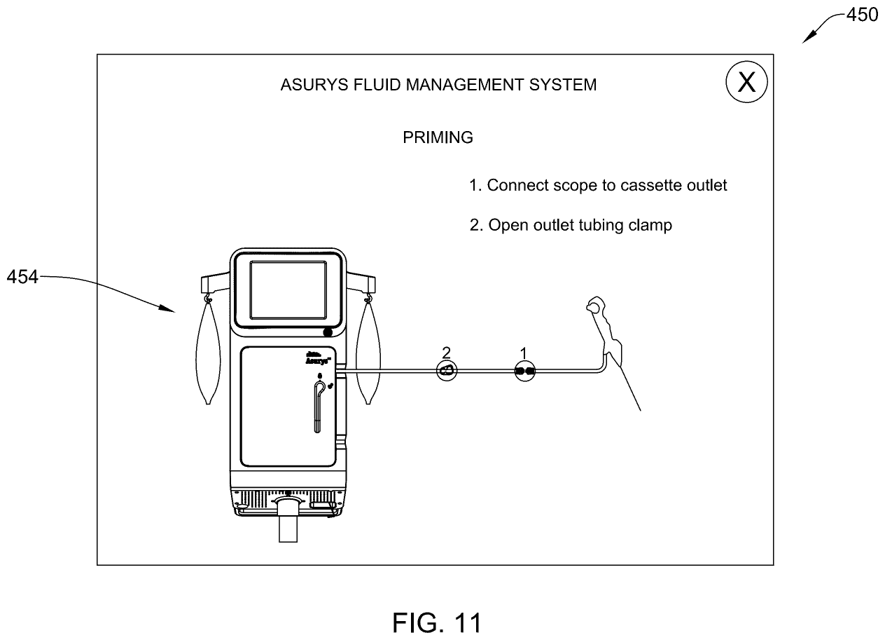

When the first stage of the priming procedure is complete, the flow path of the fluid cassette 110 may be filled with fluid from the fluid source(s). The clamp 150 on the outflow tubing 104 is closed which maintains the pressure within the fluid cassette 110 at a substantially constant stand-by pressure. The controller 20 of the fluid management system 10 may thus await initiation of the second stage of the priming procedure with the fluid cassette 110 filled with fluid at an elevated stand-by pressure. The GUI may display an initiate second stage priming screen 450, shown at FIG. 11, depicting protocol for initiating the second stage of the priming procedure. The protocol on the initiate second stage priming screen 450 may guide the user to connect an endoscope to the outflow tubing 104 and then open the clamp 150 on the outlet tubing 104. As shown in the initiate second stage priming screen 450, the screen may guide a user to sequentially connect an endoscope to the outflow tubing 104 as a first step, labeled (1), followed by opening the clamp 150, labeled (2). The initiate second stage priming screen 450 may also include an image 454 of the console and outflow tubing extending therefrom, depicting a tube of an endoscope being connected to the connector of the outflow tubing 104. The image may be labeled with a (1) near the connector, representing this step should be completed first, and the image may be labeled with a (2) near the clamp, representing this step should be completed second. In some instances, the image 454 may include an arrow pointing to the clamp in the image. In some instances, the image 454 may be animated, such that the image 454 shows movement of the tubing being connected and/or the arrow flashing at the clamp.

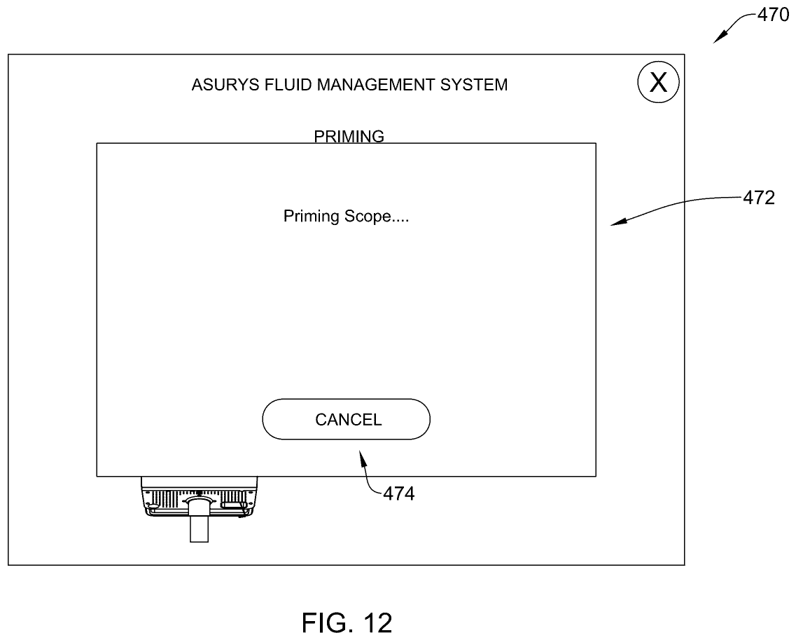

The system may automatically initiate the second stage of the priming procedure when the clamp 150 is opened by the user. To begin the second stage of the priming procedure the first stage of the priming procedure must first be successfully completed. After successful completion of the first stage of the priming procedure, the controller 30 may monitor the pump pressure of the inflow pump 60 to determine if the pressure has dropped or decreased from the pressure at the completion of the first stage of the priming procedure. If the pressure has not dropped, the controller 30 may continue to monitor the pressure of the inflow pump 60 to identify a pressure drop upon opening of the clamp 150. Opening the clamp 150 will allow fluid to flow through the outflow tubing 104 and into the endoscope (if connected) causing a drop in the pressure in the fluid cassette 110. When the pressure decreases below a predetermined threshold pressure, the controller 30 may determine that the clamp 150 has been opened and the second stage of the priming procedure should be initiated. The controller 20 detects the sudden drop in fluid pressure within the fluid cassette 110 and automatically initiates the second stage of the priming procedure. The controller may automatically initiate the second stage of the priming procedure and start the inflow pump 60 to pump fluid from the fluid source (e.g. fluid bag(s)) through the fluid cassette 110, through the outflow tubing 104 (distally past the now opened clamp 150) to the endoscope. As shown at FIG. 12, the GUI may include a Priming Scope screen 470 with a pop-up window 472 notifying the user that the second stage of the priming procedure is being performed. The pop-up window 472 may include a CANCEL button 474 which the user may use to cancel the priming procedure, if needed. This second stage of the priming procedure may purge air from the outflow tubing 104 to the endoscope, and though the infusion lumen of the endoscope. During the second stage of the priming procedure, the inflow pump 60 pulls fluid from the fluid source (e.g., fluid bag) through the fluid cassette 110 and along the outflow tubing to the endoscope, purging air from the outflow tubing 104 and the endoscope.

To initiate the second stage of the priming procedure, the motor of the inflow pump 60 may be activated and the RPMs of the inflow pump 60 increased or ramped up. The controller 30 may be configured to compare the current pressure measurement with a predetermined maximum pressure to determine if the maximum pressure has been met or exceeded. In some embodiments, the maximum pressure may be about 600 mmHg. This is just an example. The maximum pressure may be less than or greater than 600 mmHg, as desired. If the system pressure meets or exceeds the predetermined maximum pressure, the controller 30 may issue a high-pressure error. A high-pressure error may occur at any time during the second stage of the priming procedure if the measured system pressure exceeds the maximum pressure. The high-pressure error may be an alphanumeric message displayed on the touch screen interface 42, an audio alert, other visual alert, or the like. A high-pressure may be indicative of a block or obstruction in the flow path of the fluid cassette 110 or a fluid channel of the endoscope. If the pressure does not exceed a predetermined maximum, the second stage of the priming procedure may continue.

As the controller 30 is increasing or ramping the RPM of the inflow pump 60, the controller 30 may be configured to compare the current peak pressure to a predetermined threshold pressure to determine if the threshold pressure has been reached. In some cases, the threshold pressure may about 540 mmHg. This is just an example. The threshold pressure may be less than or greater than 540 mmHg, as desired. If the threshold pressure has not been reached, the controller 30 may continue to increase the RPM of the inflow pump 60.

Once the system pressure threshold has been reached, the controller 30 may increase and/or decrease the motor speed to maintain the pressure at the threshold pressure. The fluid inflow pump 60 may be maintained at the threshold pressure until at least one of two conditions is met. For example, the controller 30 may determine if a minimum or threshold volume of fluid has been delivered through the system. If the minimum or threshold volume of fluid has been delivered through the system, the second stage of the priming procedure may be considered successful and complete. The flow volume may be determined or measured using the rotations of the fluid inflow pump 60. If the minimum or threshold volume of fluid has not been delivered through the system, the fluid inflow pump 60 may continue to be maintained at the threshold pressure and the controller 30 may determine if a predetermined length of time has elapsed from the initiation of the second stage of the priming procedure. If the minimum predetermined length of time has elapsed, the second stage of the priming procedure may be considered successful and complete. If the minimum predetermined length of time has not been met or exceeded, the fluid inflow pump 60 may continue to be maintained at the threshold pressure until either the threshold volume of fluid has been delivered or the predetermined length of time has elapsed. It is contemplated that in some embodiments, the controller 30 may check the elapsed time prior to or substantially simultaneously with checking the volume of fluid delivered. In some cases, the controller 30 may be configured to select between the successful completion criteria (e.g., fluid volume or elapsed time) based on a size of the endoscope. For example, the controller 30 may rely on only one of the minimum fluid volume or minimum time elapsed to determine if the second stage of the priming procedure has been successfully completed.

It is contemplated that either the first stage or the second stage of the priming procedure may be cancelled at any time by the user via the touch screen interface 42 (e.g., the CANCEL button 404 or the CANCEL button 474). However, if the first stage of the priming procedure is not successfully completed, the fluid management system 10 may be prevented from entering a procedure mode. If the first stage of the priming procedure is cancelled, the user may be instructed to perform the first stage of the priming procedure before being able to perform the second stage of the priming procedure or before entering a procedure mode. In one example, an alphanumeric message may be displayed on the touch screen interface 42. The fluid management system 10 may be allowed to enter a procedure mode if the second stage of the priming procedure is cancelled. For example, the fluid management system 10 may enter a procedure mode upon the successful completion of the second stage of the priming procedure or upon cancellation of the second stage of the priming procedure. As described above, if the second stage of the priming procedure is not initialized within the predetermined length of time (e.g., 60 minutes) following the completion of the first stage of the priming procedure, the user may be instructed to perform the first stage of the priming procedure again.

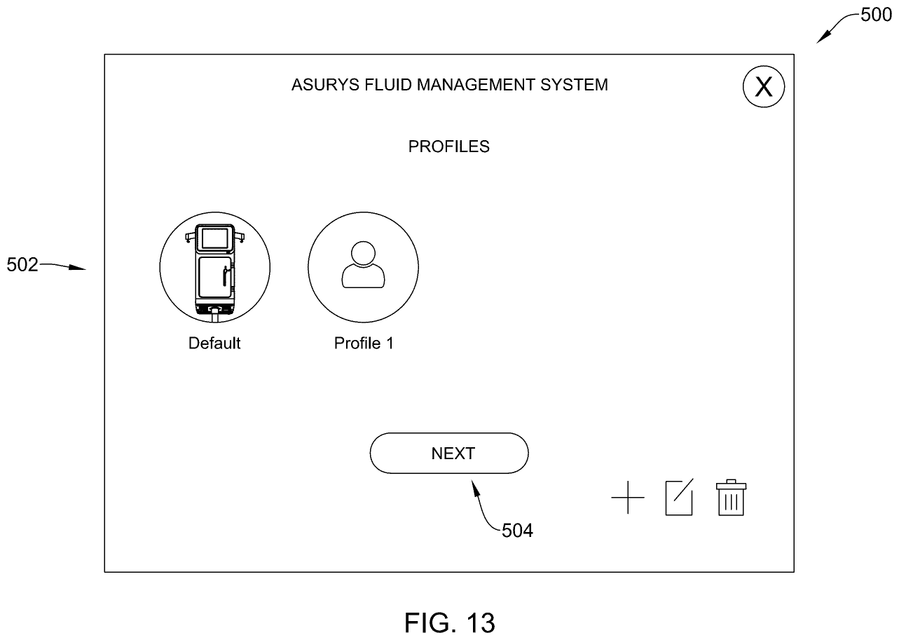

At the completion of the priming procedure, the Profile screen 500 of the GUI, shown at FIG. 13, may be displayed for selection of a user profile associated with a medical procedure to be performed with the fluid management system 10. The Profile screen 500 may also be accessible through selection of the Profile icon 202 from the Home Screen 200. The Profile screen 500 permits the creation, editing, deletion of multiple user profiles 502. Once a desired profile has been selected, the user may select the NEXT button 504 to move to the next screen of the step-by-step setup of the GUI.

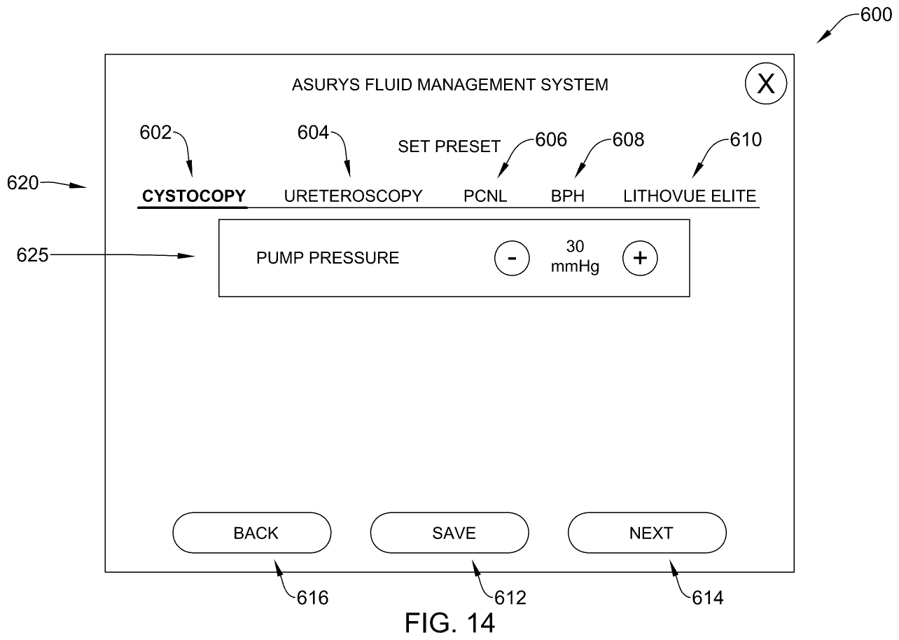

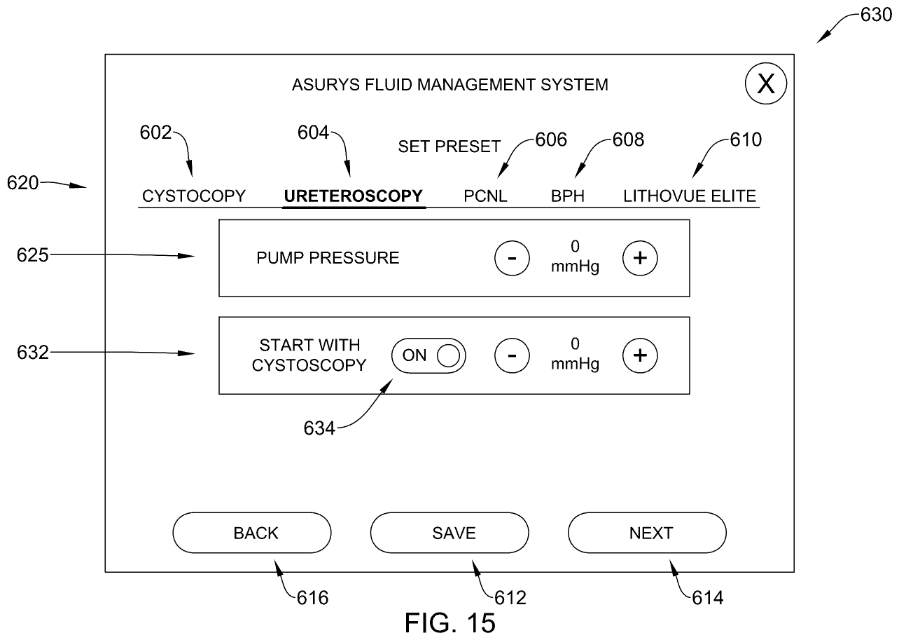

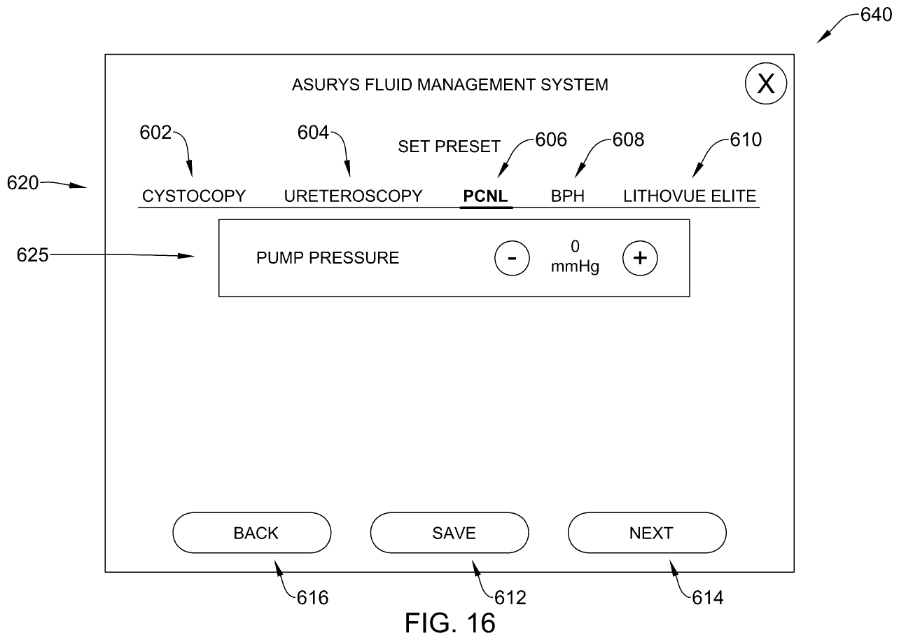

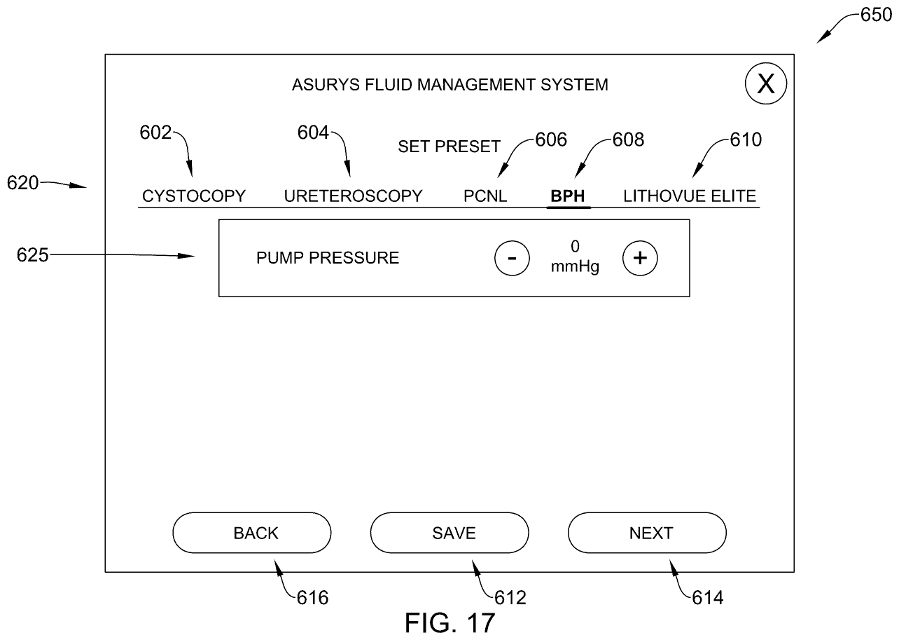

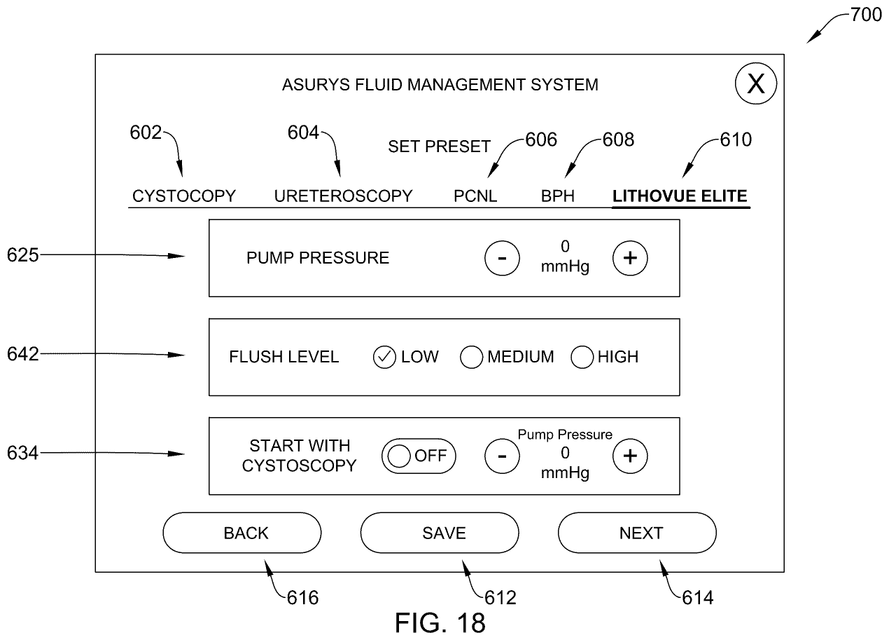

The next screen permits the user to select one of several medical procedure modes from a Medical Procedure Mode screen from the GUI shown in FIGS. 14-19. For example, several procedure modes may be displayed in a procedure menu 620 across the display 44 for user selection. The procedure modes may include a Cystoscopy mode screen 600 in which the Cystoscopy input 602 has been selected by the user (e.g., via tapping the Cystoscopy input 602 portion of the display 44), shown in FIG. 14 for initiating a cystoscopy procedure, a Ureteroscopy mode screen 630 in which the Ureteroscopy input 604 has been selected by the user (e.g., via tapping the Ureteroscopy input 604 portion of the display 44), shown in FIG. 15 for initiating a ureteroscopy procedure, a PCNL mode screen 640 in which the PCNL input 606 has been selected by the user (e.g., via tapping the PCNL input 606 portion of the display 44), shown in FIG. 16 for initiating a PCNL (percutaneous nephrolithotomy) procedure, a BPH mode screen 650 in which the PBH input 608 has been selected by the user (e.g., via tapping the BPH input 608 portion of the display 44), shown in FIG. 17 for initiating a BPH (benign prostatic hyperplasia) procedure, or a Lithovue Elite mode screen 700 in which the Lithovue Elite input 610 has been selected by the user (e.g., via tapping the Lithovue Elite input 610 portion of the display 44), shown in FIG. 18 for initiating a procedure utilizing a LithoVue™ Elite endoscope having a pressure sensor at the distal end of the endoscope, or other intraluminal pressure sensing endoscope, that provides real-time intraluminal pressure to the fluid management system 10 via the pressure sensor provided with the endoscope. The Lithovue Elite mode screen 700 is a mode screen that is only active/permitted when the fluid management system 10 recognizes a Lithovue™ Elite endoscope has been connected to the fluid management system 10 for use therewith.

When the Cystoscopy input 602 is selected, the GUI displays the Cystoscopy settings screen 600 to the user, shown in FIG. 14. The Cystoscopy settings screen 600 permits the user to set the desired pump pressure setpoint that the fluid management system 10 will maintain during the medical procedure. For example, a pump pressure window 625 will be displayed, allowing the user to increase and/or decrease the setting for the pump pressure setpoint to a desired target pump pressure. Once the desired pump pressure setpoint is set and displayed, the user may save the set pump pressure setpoint by selecting the SAVE button 612. Once the desired pump pressure setpoint is saved, the user may select the NEXT button 614 to move to the next screen of the GUI, which is the intraoperative screen displayed during the medical procedure. In the event the user wants to return to a previous screen of the step-by-step setup of the GUI, the user may select the BACK button 616.

When the Ureteroscopy input 604 is selected, the GUI displays the Ureteroscopy settings screen 630 to the user, shown in FIG. 15. The Ureteroscopy settings screen 630 permits the user to set the desired pump pressure setpoint that the fluid management system 10 will maintain during the medical procedure. For example, a pump pressure window 625 will be displayed, allowing the user to increase and/or decrease the setting for the pump pressure setpoint to a desired target pump pressure. Once the desired pump pressure setpoint is set and displayed, the user may save the set pump pressure setpoint by selecting the SAVE button 612.

The Ureteroscopy settings screen 630 also presents a Start With Cystoscopy window 632 to the user, permitting the user to select whether it is desired to begin the ureteroscopy procedure with cystoscopy. For example, the Start With Cystoscopy window 632 may include a user input 634 to selectively select or deselect Start With Cystoscopy (e.g., turn Start With Cystoscopy “on” or “off”). In the event the user would like to start with cystoscopy and turns the Start With Cystoscopy input 634 to “on”, the user is able to increase and/or decrease the setting for the pump pressure to a desired target pump pressure setpoint for use in the cystoscopy portion of the medical procedure. Thus, the pump pressure setting (i.e., target pump pressure set point) for use during the cystoscopy portion of the medical procedure may be set independent of, and thus may be different from, the pump pressure setting (i.e., target pump pressure set point) for use during the ureteroscopy portion of the medical procedure. Accordingly, when the inflow pump 60 is turned on at the beginning of a medical procedure (e.g., ureteroscopy procedure) in which a cystoscope is initially fluidly attached to the fluid management system 10, the inflow pump 60 will run at a desired speed to maintain the pump pressure setting set for the cystoscopy portion of the medical procedure from the pump pressure setting of window 632. At the conclusion of the cystoscopy portion, and thus the beginning of the ureteroscopy portion of the medical procedure, the fluid management system 10 will change to operating the inflow pump 60 at a desired speed to maintain the pump pressure setting set for the ureteroscopy portion from the pump pressure window 625. A ureteroscope may be exchanged for the cystoscope during the transition between the cystoscopy portion of the medical procedure and the ureteroscopy portion of the medical procedure, such that the ureteroscope is fluidly coupled to the fluid management system 10 for delivery of fluid therethrough. A user may select an input from the GUI indicating that the user wishes to end the cystoscopy portion and begin the ureteroscopy portion of the medical procedure, thus adjusting the pump pressure setting that the inflow pump 60 is intended to maintain.

Once the desired pump pressure setpoint is saved and the user has indicated whether the medical procedure is to begin with cystoscopy at the pump pressure setpoint provided at window 632, the user may select the NEXT button 614 to move to the next screen of the GUI, which is the intraoperative screen displayed during the medical procedure. In the event the user wants to return to a previous screen of the step-by-step setup of the GUI, the user may select the BACK button 616.

When the PCNL input 606 is selected, the GUI displays the PCNL settings screen 640 to the user, shown in FIG. 16. The PCNL settings screen 640 permits the user to set the desired pump pressure setpoint that the fluid management system 10 will maintain during the medical procedure. For example, a pump pressure window 625 will be displayed, allowing the user to increase and/or decrease the setting for the pump pressure setpoint to a desired target pump pressure. Once the desired pump pressure is set and displayed, the user may save the set pump pressure setpoint by selecting the SAVE button 612. Once the desired pump pressure setpoint is saved, the user may select the NEXT button 614 to move to the next screen of the GUI, which is the intraoperative screen displayed during the medical procedure. In the event the user wants to return to a previous screen of the step-by-step setup of the GUI, the user may select the BACK button 616.

When the BPH input 608 is selected, the GUI displays the BPH settings screen 650 to the user, shown in FIG. 17. The BPH settings screen 650 permits the user to set the desired pump pressure setpoint that the fluid management system 10 will maintain during the medical procedure. For example, a pump pressure window 625 will be displayed, allowing the user to increase and/or decrease the setting for the pump pressure setpoint to a desired target pump pressure. Once the desired pump pressure setpoint is set and displayed, the user may save the set pump pressure setpoint by selecting the SAVE button 612. Once the desired pump pressure is saved, the user may select the NEXT button 614 to move to the next screen of the GUI, which is the intraoperative screen displayed during the medical procedure. In the event the user wants to return to a previous screen of the step-by-step setup of the GUI, the user may select the BACK button 616.

When the Lithovue Elite input 610 is selected, the GUI displays the Lithovue Elite mode screen 700 to the user, shown in FIG. 18. The Lithovue Elite mode screen 700 permits the user select or set one or more settings, such as an input to set the desired pump pressure setpoint that the fluid management system 10 will maintain during the medical procedure. For example, a pump pressure window 625 will be displayed, allowing the user to increase and/or decrease the setting for the pump pressure setpoint to a desired target pump pressure. Once the desired pump pressure setpoint is set and displayed, the user may save the set pump pressure setpoint by selecting the SAVE button 612.

The Lithovue Elite mode screen 700 also presents a Flush Level setting window 642 to the user, permitting the user to select the flush level desired when the flush button is activated on the fluidly attached endoscope (e.g., on the LithoVue™ Elite endoscope) by the user. The Flush Level setting window 642 may allow the user to select one of a plurality of different flush levels, such as a “low” flush level, a “medium” flush level, and a “high” flush level.

The Lithovue Elite mode screen 700 also presents a Start With Cystoscopy window 632 to the user, permitting the user to select whether it is desired to begin the endoscopic procedure with cystoscopy. For example, the Start With Cystoscopy window 632 may include a user input 634 to selectively select or deselect Start With Cystoscopy (e.g., turn Start With Cystoscopy “on” or “off”). In the event the user would like to start with cystoscopy and turns the Start With Cystoscopy input 634 to “on”, the user is able to increase and/or decrease the setting for the pump pressure setpoint to a desired target pump pressure for use in the cystoscopy portion of the medical procedure. Thus, the pump pressure setting (i.e., target pump pressure set point) for use during the cystoscopy portion of the medical procedure may be set independent of, and thus may be different from, the pump pressure setting (i.e., target pump pressure set point) for use during the other endoscopic portion (e.g., ureteroscopy portion) of the medical procedure. Accordingly, when the inflow pump 60 is turned on at the beginning of a medical procedure (e.g., ureteroscopy procedure) in which a cystoscope is initially fluidly attached to the fluid management system 10, the inflow pump 60 will run at a desired speed to maintain the pump pressure setpoint setting set for the cystoscopy portion of the medical procedure from the pump pressure setting of window 632. At the conclusion of the cystoscopy portion, and thus the beginning of the ureteroscopy portion of the medical procedure in which the LithoVue™ Elite endoscope, or other intraluminal pressure sensing endoscope, is used, the fluid management system 10 will change to operating the inflow pump 60 at a desired speed to maintain the pump pressure setpoint setting set for the ureteroscopy portion from the pump pressure window 625. The Litho Vue™ Elite endoscope may be exchanged for the cystoscope during the transition between the cystoscopy portion of the medical procedure and the ureteroscopy portion of the medical procedure, such that the LithoVue™ Elite endoscope is fluidly coupled to the fluid management system 10 for delivery of fluid therethrough. A user may select an input from the GUI indicating that the user wishes to end the cystoscopy portion and begin the ureteroscopy portion of the medical procedure, thus adjusting the pump pressure setpoint setting that the inflow pump 60 is intended to maintain.

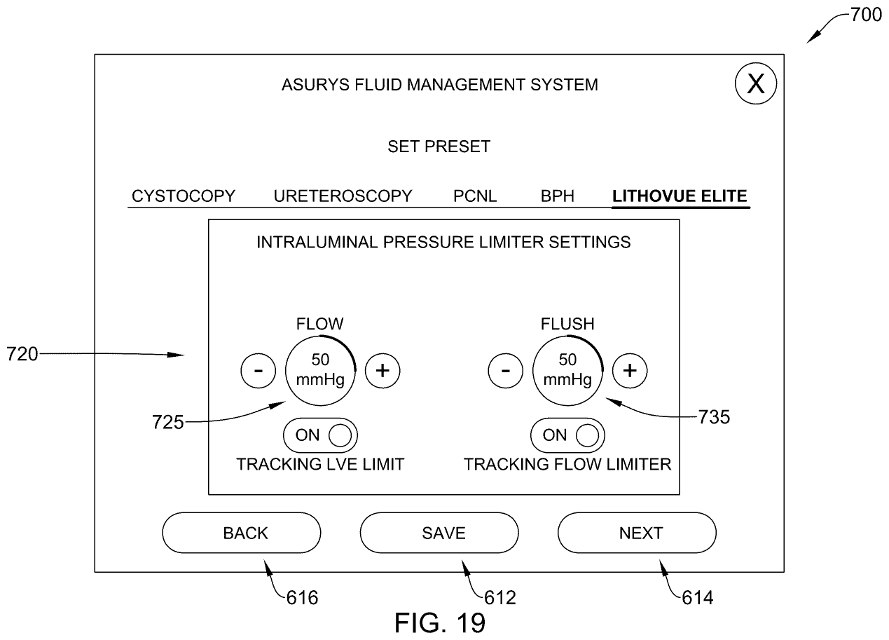

Once the desired settings have been made, the user may save the settings by selecting the SAVE button 612. In the event the user wants to return to a previous screen of the step-by-step setup of the GUI, the user may select the BACK button 616. Once the desired pump pressure setting is saved, the desired flush level is selected, and the user has indicated whether the medical procedure is to begin with cystoscopy at the pump pressure setpoint provided at window 632, the user may select the NEXT button 614 to move to the next screen of the GUI, in which the Intraluminal Pressure Limiter Settings window 720 is displayed on the display 44, shown at FIG. 19.

The Intraluminal Pressure Limiter Settings window 720 includes a flow pressure limiter input 725 that allows the user to input/adjust a desired pressure limit for the intraluminal pressure limiter for fluid flow through the attached endoscope during a medical procedure. The set pressure limit for the intraluminal pressure limiter for fluid flow may be displayed on the display 44 or another display connected to the intraluminal pressure sensing endoscope (e.g., Litho Vue™ Elite endoscope) during the medical procedure. The Intraluminal Pressure Limiter Settings window 720 includes a flush pressure limiter input 735 that allows the user to input/adjust a desired pressure limit for the intraluminal pressure limiter for fluid flush through the attached endoscope during a medical procedure when a fluid flush is activated. The set pressure limit for the intraluminal pressure limiter for fluid flush may be displayed on the display™ 44 or another display connected to the intraluminal pressure sensing endoscope (e.g., Litho Vue′ Elite endoscope) during the medical procedure.

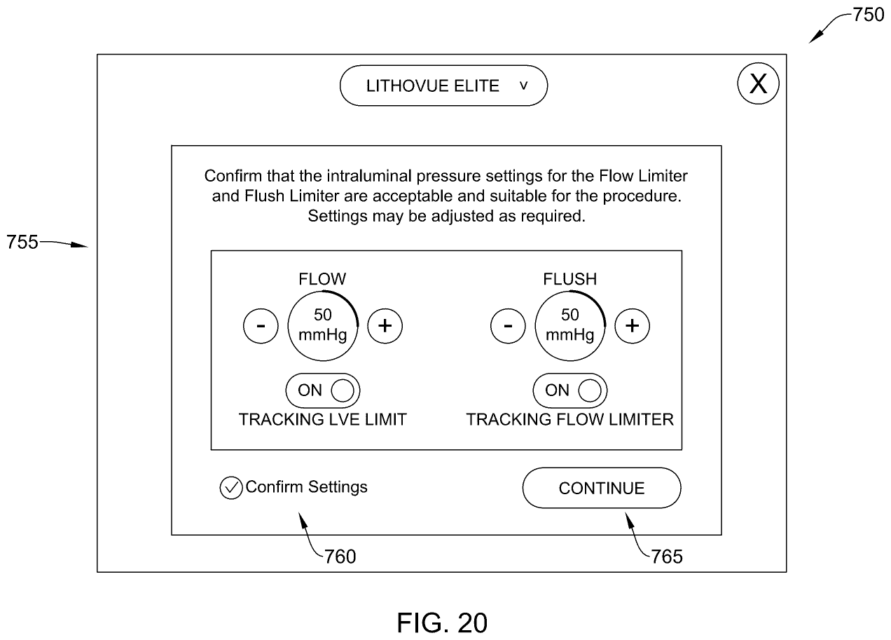

Once the desired intraluminal pressure limiter settings (for fluid flow and fluid flush) are set and displayed, the user may save the settings by selecting the SAVE button 612. In the event the user wants to return to a previous screen of the step-by-step setup of the GUI, the user may select the BACK button 616. Once the desired intraluminal pressure limiter settings are saved, the user may select the NEXT button 614 to move to the next screen of the GUI, which is Intraluminal Pressure Limiter Settings Confirmation screen 750, shown at FIG. 20. The Intraluminal Pressure Limiter Settings Confirmation screen 750 includes a confirmation window 755 including text asking the user to confirm that the input/set pressure settings from the preceding Intraluminal Pressure Limiter Settings window 720 are acceptable and suitable for the medical procedure to be performed. The confirmation window 755 may require a multiple step confirmation process (e.g., a two-step confirmation process) to confirm that the input/set pressure settings from the preceding Intraluminal Pressure Limiter Settings window 720 are acceptable and suitable for the medical procedure to be performed. For example, the user may be required first to select (e.g., tap) a first or primary confirmation indicator 760 (e.g., graphical and/or alphanumeric icon) in which the user is acknowledging to the controller of the fluid management system 10 that the input/set pressure settings from the preceding Intraluminal Pressure Limiter Settings window 720 are acceptable and suitable for the medical procedure to be performed. Once the first confirmation indicator 760 has been selected, the user must then select (e.g., tap) a second or secondary confirmation indicator 765 (e.g. graphical and/or alphanumeric icon) in which the user acknowledges that the user is ready to proceed with the medical procedure. For example, the second or secondary confirmation indicator 765 may be a CONTINUE or START button provided on the confirmation window 755.

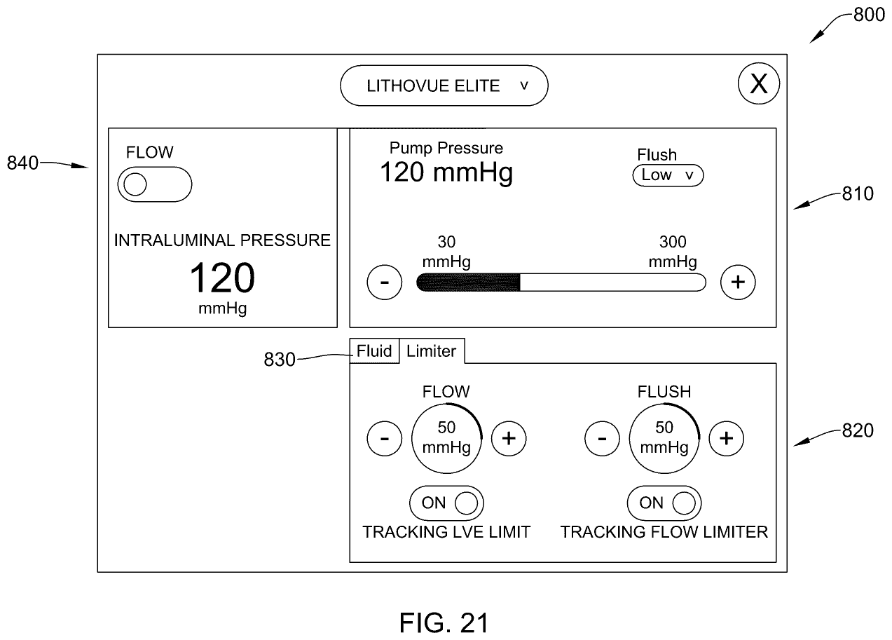

Once the user has selected the second or secondary confirmation indicator 765 the GUI may display a Live or intraoperative screen 800, shown at FIG. 21, on the display 44 and initiate the medical procedure. For example, the inflow pump 60 may be activated and fluid may begin flowing through the fluid management system 10 to the connected endoscope. The Live or intraoperative screen 800 may display one or more, or a plurality of settings, parameters, and/or monitored data. For example, the Live or intraoperative screen 800 may include a pump pressure window 810 displaying the pump pressure setpoint and/or the currently measured pump pressure. The pump pressure window 810, or a separate window, may also display the set flush level and/or an icon indicating whether the flush function is activated or not activated. The pump pressure window 810 may also include an input to intraoperatively adjust the pump pressure setpoint, in some instances.

The Live or intraoperative screen 800 may also include a pressure limiter window 820 displaying the flow pressure limiter settings and/or the flush pressure limiter settings. The pressure limiter window 820 may also include an input to intraoperatively adjust the flow pressure limiter settings and/or the flush pressure limiter settings, in some instances.

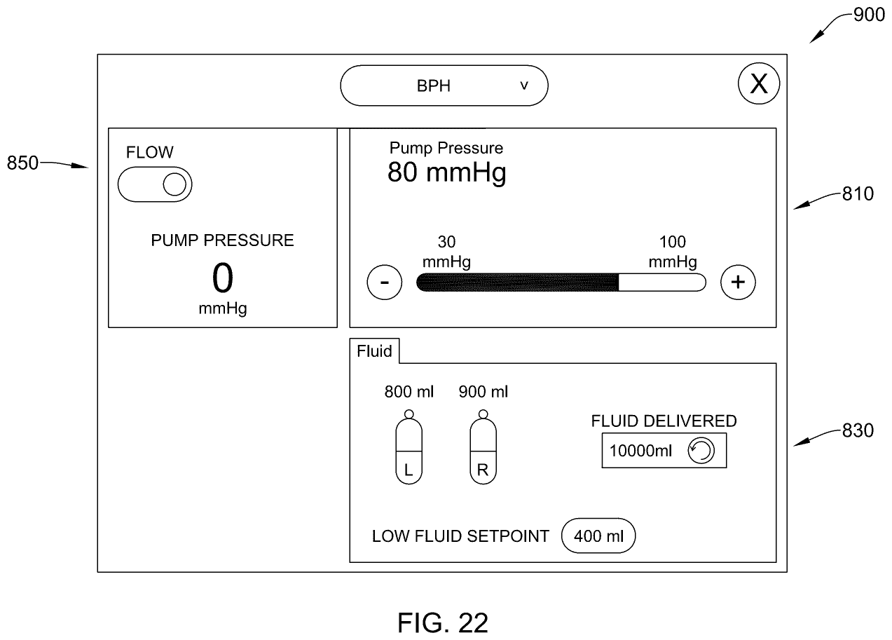

The Live or intraoperative screen 800 may also include a fluid level window 830, see FIG. 22, displaying the fluid level in the fluid bag(s) hanging from the fluid supply source hanger(s) 32. The fluid level window 830 may also include a counter for displaying the total amount of fluid delivered through the fluid management system 10 during a medical procedure. In some instances, the total amount of fluid delivered may be calculated based on the change in weight of the fluid bag(s) and/or the number of revolutions of the inflow pump 60 completed during the medical procedure. The fluid level window 830 may also include an input to intraoperatively set/adjust the low fluid setpoint (e.g., the threshold level at which the fluid management system alerts the user to an occurrence that the total amount of fluid in the fluid bag(s) has decreased below a threshold level), in some instances.

In some instances, the user may be able to toggle or tab between the pressure limiter window 820 and the fluid level window 830 from the Live or intraoperative screen 800, such that only one of the pressure limiter window 820 and the fluid level window 830 is displayed at any one time.

The Live or intraoperative screen 800 may also include an intraluminal pressure window 840 displaying the current, real-time intraluminal pressure sensed by the pressure sensor on the intraluminal pressure sensing endoscope (e.g., LithoVue™ Elite endoscope) during the medical procedure.

FIG. 22 depicts an alternative Live or intraoperative screen 900 that may be displayed on the display 44 when a medical procedure is initiated from any one of the several medical procedure modes not utilizing an intraluminal pressure sensing endoscope. For example, the GUI may enter the Live or intraoperative screen 900 upon selecting the NEXT button 614 from any one of the Cystoscopy settings screen 600 shown in FIG. 14, the Ureteroscopy settings screen 630 shown in FIG. 15, the PCNL settings screen 640 shown in FIG. 16, or the BPH settings screen 650 shown in FIG. 17. At this point, the inflow pump 60 may be activated and fluid may begin flowing through the fluid management system 10 to the connected endoscope.

The Live or intraoperative screen 900 may display one or more, or a plurality of settings, parameters, and/or monitored data. For example, the Live or intraoperative screen 900 may include a pump pressure window 810 displaying the pump pressure setpoint and/or the currently measured pump pressure. The pump pressure window 810 may also include an input to intraoperatively adjust the pump pressure setpoint, in some instances. In some instances, the Live or intraoperative screen 900 may include a second pump pressure window 850 displaying the currently measured pump pressure, while the first pump pressure window 810 may display the pump pressure setpoint and include an input to intraoperatively adjust the pump pressure setpoint.

The Live or intraoperative screen 900 may also include a fluid level window 830 displaying the fluid level in the fluid bag(s) hanging from the fluid supply source hanger(s) 32. The fluid level window 830 may also include a counter for displaying the total amount of fluid delivered through the fluid management system 10 during a medical procedure. In some instances, the total amount of fluid delivered may be calculated based on the change in weight of the fluid bag(s) and/or the number of revolutions of the inflow pump 60 completed during the medical procedure. The fluid level window 830 may also include an input to intraoperatively set/adjust the low fluid setpoint (e.g., the threshold level at which the fluid management system alerts the user to an occurrence that the total amount of fluid in the fluid bag(s) has decreased below a threshold level), in some instances.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system, the system comprising:
a fluid management console, comprising:
a housing;
a controller housed within the housing;
an inflow pump disposed within the housing; and
a graphical user interface (GUI) associated with the housing;
a fluid cassette configured to be received within a receptacle of the housing of the fluid management console; and
wherein the GUI includes a series of start-up screens including a fluid priming screen prompting a user to select between a quick setup mode and a step-by-step setup mode in order to complete a priming procedure of the fluid cassette.

2. The fluid management system of claim 1, wherein the quick setup mode includes a screen listing a series of steps to be performed to complete the priming procedure.

3. The fluid management system of claim 2, wherein each of the series of steps must be confirmed prior to completing the priming procedure.

4. The fluid management system of claim 3, wherein the controller confirms each of the series of steps with one or more sensors.

5. The fluid management system of claim 4, wherein the one or more sensors include one or more proximity sensors, one or more contact sensors, and/or one or more magnetic sensors.

6. The fluid management system of claim 1, wherein the quick setup mode includes a user input to confirm that a clamp on a fluid outlet tubing extending from the fluid cassette has been closed.

7. The fluid management system of claim 6, wherein confirmation that the clamp on the fluid outlet tubing is closed must be completed prior to the controller initiating the priming procedure.

8. The fluid management system of claim 1, wherein the step-by-step setup mode includes one or more screens of the GUI depicting an image of:
a) opening the door the console;
b) installing the fluid cassette within the receptacle of the housing; or
c) closing the door.

9. The fluid management system of claim 8, wherein the image is animated.

10. The fluid management system of claim 8, wherein the step-by-step setup mode includes a user input to confirm that a clamp on a fluid outlet tubing extending from the fluid cassette has been closed.

11. A fluid management system, the system comprising:
a fluid management console, comprising:
a housing;
a controller housed within the housing;
an inflow pump disposed within the housing; and
a graphical user interface (GUI) associated with the housing;
wherein the GUI includes a plurality of medical procedure mode screens for setting one or more parameters for controlling the inflow pump, the plurality of medical procedure mode screens including a first mode for use of the fluid management system with a pressure sensing endoscope having a pressure sensor incorporated therewith for communicating intraluminal pressure to the controller, and one or more modes for use of the fluid management system with an endoscope without a pressure sensor.

12. The fluid management system of claim 11, wherein the plurality of medical procedure mode screens includes a cystoscopy mode and a ureteroscopy mode.

13. The fluid management system of claim 12, wherein upon selecting the ureteroscopy mode, the GUI includes a prompt and/or window permitting the user to start the medical procedure in the cystoscopy mode.

14. The fluid management system of claim 13, wherein the controller includes different user selectable pump pressure settings in the cystoscopy mode than in the ureteroscopy mode.

15. The fluid management system of claim 11, wherein upon selecting the first mode, the GUI includes a prompt and/or window permitting the user to start the medical procedure in a cystoscopy mode.

16. The fluid management system of claim 15, wherein the controller includes different user selectable pump pressure settings in the cystoscopy mode than in the first mode.

17. A fluid management system, the system comprising:
a fluid management console, comprising:
a housing;
a controller housed within the housing;
an inflow pump disposed within the housing; and
a graphical user interface (GUI) associated with the housing;
wherein the GUI includes a medical procedure mode screen for setting one or more parameters for controlling the inflow pump for use of the fluid management system with a pressure sensing endoscope having a pressure sensor incorporated therewith;
wherein the GUI includes a screen to input and/or set intraluminal pressure limiter settings into the controller.

18. The fluid management system of claim 17, wherein the GUI includes a confirmation window requiring a multiple step confirmation process to confirm that the input/set intraluminal pressure limiter settings are acceptable.

19. The fluid management system of claim 18, wherein the GUI includes an intraoperative screen permitting a user to adjust the intraluminal pressure limiter settings during a medical procedure.

20. The fluid management system of claim 17, wherein the intraluminal pressure limiter settings include an intraluminal pressure limiter setting for fluid flow through an attached endoscope and an intraluminal pressure limiter setting for fluid flush through the attached endoscope a fluid flush is activated by the attached endoscope, the intraluminal pressure limiter setting for fluid flow being independent of the intraluminal pressure limiter setting for fluid flush.
